(19) United States
(12) Patent Application Publication
(10) Pub. No.: US 2008/0074619 A1
(43) Pub. Date: Mar. 27, 2008

(54) Reading glass strenght self-tester

(76) Inventor: Andrew Gisonna, Oakland, NJ (US)

Correspondence Address: HOGAN & HARTSON L.L.P., 875 THIRD AVENUE, NEW YORK, NY, 10022, US

(21) Appl. No.: 11/879,591

(22) Filed: Jul. 18, 2007

Related U.S. Application Data
(60) Provisional application No. 60/831,507, filed on Jul. 18, 2006.

(51) Int. Cl. A61B 3/04

(52) U.S. Cl. 351/223

(57) ABSTRACT

A reading glass strength self-tester comprising a housing that incorporates a viewport in the front of the housing. Magnifying lens of different diopter ratings are mounted behind the viewport to magnify a reading surface located within the housing. A window incorporated into the front of the housing displays the diopter ratings of the lenses. The lenses can be advanced by the user behind the viewport for viewing by the user. At least one mirror located behind the lenses directs the image on the reading surface along a non-linear viewing path in relation to the viewport. A non-ambient light source mounted inside the housing illuminates the reading surface. A power source is connected to the light source by electrical means, which can be turned on by the user. The user can use the self-tester to determine the appropriate strength of non-prescription reading glasses required to improve or correct the consumer's farsightedness by magnifying images reaching the eye.

### CROSS REFERENCE TO RELATED APPLICATIONS

This application claims priority to U.S. Provisional Application No. 60/831,507 filed Jul. 18, 2006, which application is expressly incorporated herein by reference in its entirety.

### BACKGROUND OF THE INVENTION

1. Field of the Invention

The invention relates generally to a vision testing device. More particularly, the invention relates to a standalone testing device having its own source of light for use by a consumer for testing near vision to determine the appropriate strength of non-prescription reading glasses required to improve or correct the consumer's farsightedness by magnifying images reaching the eye.

2. Relevant Background

Farsightedness, also known as hyperopia, affects many people and is common with age. Farsighted people have difficulty reading or seeing near objects. In the normal eye, light rays coming into the eye converge into a focal point at the retina. The retina then sends a clear image to the brain. But in those afflicted with farsightedness, a shorter distance from cornea to retina causes light rays to pass the retina before coming to a focal point. This results in a blurred image being sent to the brain. Farsightedness may be easily improved with widely available non-prescription reading glasses.

As is described in U.S. Pat. Nos. 5,861,941, 5,486,879 and 6,257,724, non-prescription reading glasses are commonly sold directly to consumers at pharmacies and other retail outlets. Such non-prescription reading glasses are often displayed in a retail establishment on free-standing point-of-sale displays.

Typically, a consumer selects a pair of non-prescription reading glasses from the display by trying on a number of pairs until he locates a pair that is suitable, from the standpoint of comfort, magnifying ability and appearance. In order to determine the appropriate magnifying ability (measured in terms of diopters) for the reading glasses, eye charts are often placed on the reading glass display. The charts have increasing sizes of print, and the consumer is instructed to stand approximately 14 inches (35.6 cm.) from the chart. When viewing the chart, the consumer will look at each line of print, and note the first line of print that appears to be out of focus. By reading across the chart, the consumer can then determine the amount of magnification needed for the reading glasses.

Once the consumer determines the appropriate amount of magnification needed, the consumer will then view all of the glasses on the display rack that have lenses of the appropriate diopter rating. The consumer can then select a pair of reading glasses that the consumer finds to be attractive and comfortable.

Although the use of eye charts can effectively determine the proper amount of magnification needed for the reading glasses, as described in the '941 and '724 patents, their use can be imprecise. Quite often, the consumer will not stand an appropriate distance from the chart. Alternatively, consumers may test reading glasses by simply trying on a large number of reading glasses, of different magnifications, and testing them in connection with reading matter that is hand-held by the consumer.

In an attempt to overcome these problems, the '879 patent describes a hand-held vision tester comprising a closed tunnel having at one end two plates, which sandwich two discs containing a series of magnifying lenses. At the other end of the tunnel is a light permeable plate containing letters, numbers or other visual indicia for the consumer to view. The consumer rotates the magnifying discs until the visual indicia are in focus for each eye, and then determines the corresponding diopter.

Similarly, the '941 patent describes a vision tester incorporated into a point-of-sale display. The vision tester of the '941 patent includes front and back walls approximately 14 inches (35.6 cm) apart. Visual indicia are printed on the back wall. The front wall contains an opening through which the consumer views the print. Magnifying lenses of varying strengths are positionable behind the opening, and by changing lenses, the consumer can determine the optimal diopter to improve or correct his farsightedness. But according to the later '724 patent, the '941 patent suffers from several disadvantages, foremost of which is the fact that the tester of '724 patent relies on indirect ambient light to illuminate the printed matter. To overcome that problem, the '941 patent describes a similar tester, but which has an indirect view path to permit direct illumination of the print surface. The vision tester of the '724 patent includes front and top walls oriented at an angle of 90° to one another. The top wall containing the visual indicia is oriented parallel to the ceiling of the store such that ambient light enters it directly from above to illuminate the printed matter. A mirror is positioned at an angle between the front and top walls to reflect the printed image toward the viewer. Although the view path is indirect, the effective vision path is again approximately 14 inches (35.6 cm).

An obvious shortcoming of all of the testing devices described above is their reliance on ambient light to illuminate the visual indicia. In addition, the testers of the '724 and '941 patents are not standalone devices, but are instead incorporated into a point-of-sale display.

In view of the foregoing, it is an object of this invention to provide a simple, easy-to-use reading glass strength self-tester that includes its own source of light.

It is as another object of the invention to provide a reading glass strength self-tester that is small, portable and does not require incorporation into a point-of-purchase display (although it may be).

It is a further object of the invention to provide a reading glass strength self-tester that does not require the customer to rely on or use the saleable product (i.e. actual reading glasses being offered for sale) to conduct the test.

### SUMMARY OF THE INVENTION

The invention relates generally to a vision testing device. More particularly, the invention relates to a standalone testing device having its own source of light for use by a consumer for testing near vision to determine the appropriate strength of non-prescription reading glasses required to improve or correct the consumer's farsightedness by magnifying images reaching the eye.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 is a front elevation view of a reading glass strength tester according to one embodiment of the invention.

FIG. 2 is a side elevation view of the reading glass strength tester of FIG. 1.

FIG. 3 is a rear elevation view of the reading glass strength tester of FIG. 1.

FIGS. 4 and 5 are front side perspective views of the reading glass strength tester of FIG. 1.

FIG. 6 is a perspective view of a housing and adjustment knob according to one embodiment of the invention, partially cut away to show the lens carrier.

FIG. 7 is a perspective view of the right half of the housing of FIG. 6 in which the adjustment knob is not shown.

FIG. 7A is a perspective view of FIG. 6, partially cut away to show the rack and pinion system for advancing the lens carrier.

FIGS. 8 and 9 are side and front perspective views of a reading glass strength tester according to the invention.

FIGS. 8a and 9a are perspective views of FIGS. 8 and 9, respectively, cut away to show the lens carrier assembly, the view path, the light source and the power source.

FIG. 10 is a perspective view of a portion of the lens carrier assembly shown in FIGS. 8a and 9a.

FIG. 11 is a front elevation view of the portion of the lens carrier assembly shown in FIGS. 8a and 9a.

FIG. 12 is an elevation view of a magnifying lens that may be disposed in the lens carrier of FIGS. 10 and 11.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein.

The invention relates generally to a vision testing device. More particularly, the invention relates to a standalone testing device having its own source of light for use by a consumer for testing near vision to determine the appropriate strength of non-prescription reading glasses required to improve or correct the consumer's farsightedness.

The reading glass strength tester is intended to aid an individual in choosing the correct diopter or magnification of lens for non-prescription reading glasses. As newsprint is sometimes difficult to read because of the font type and size, an individual may choose to wear reading glasses with magnification lenses. These lenses do not correct poor vision; they merely magnify the images being viewed.

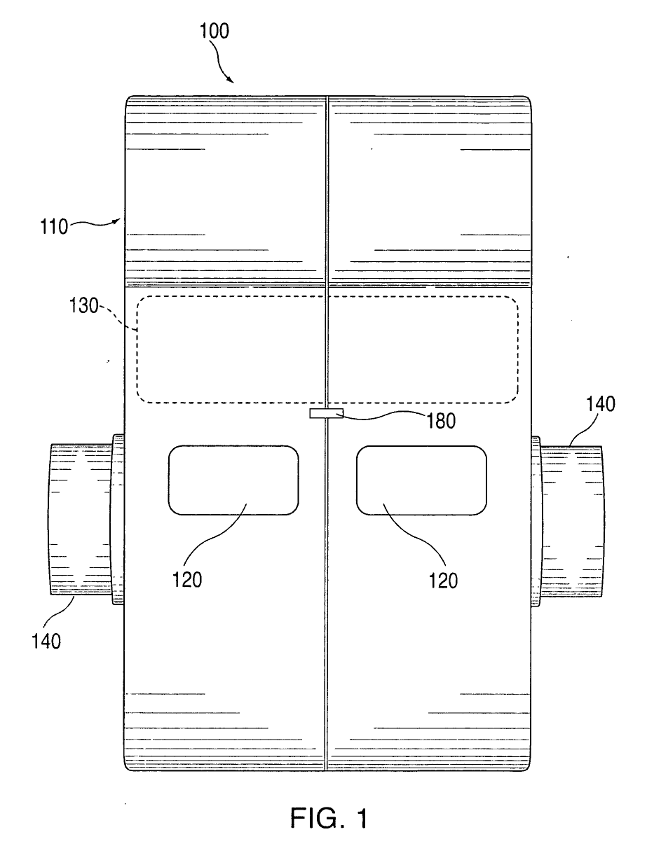

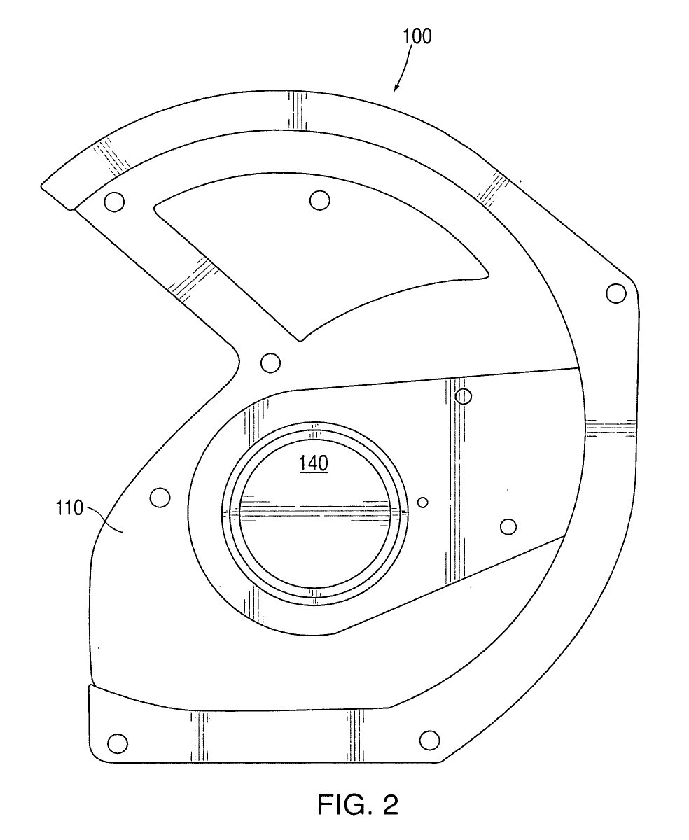

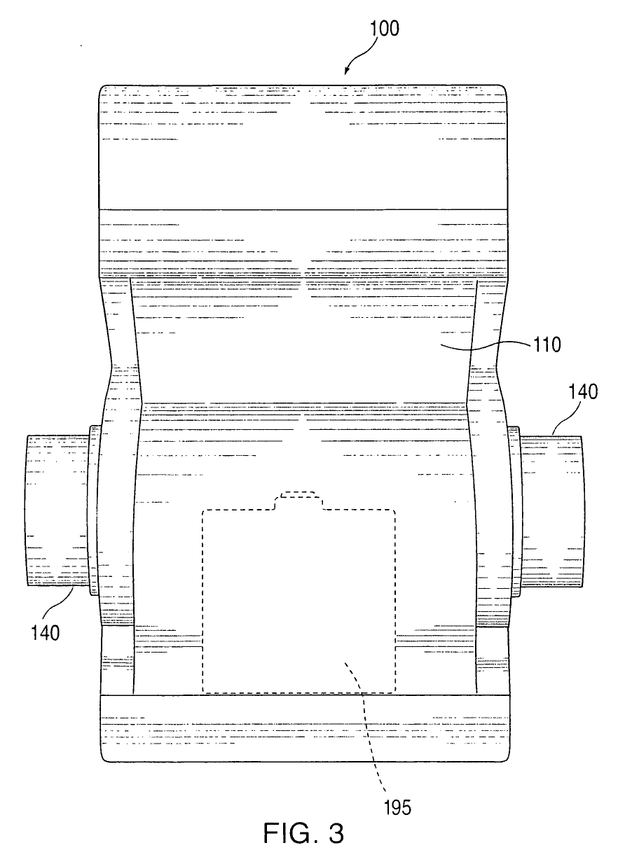

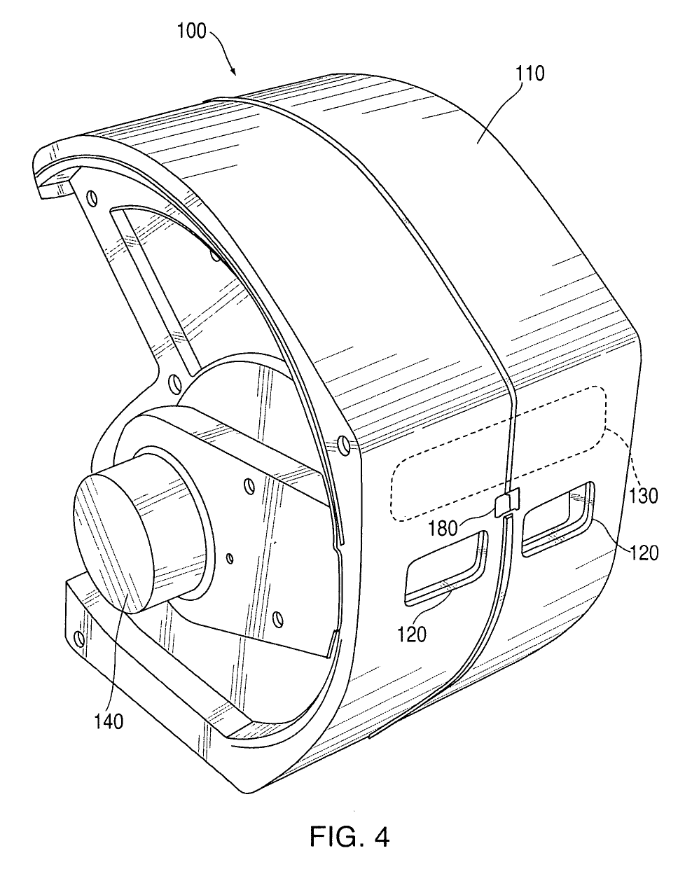

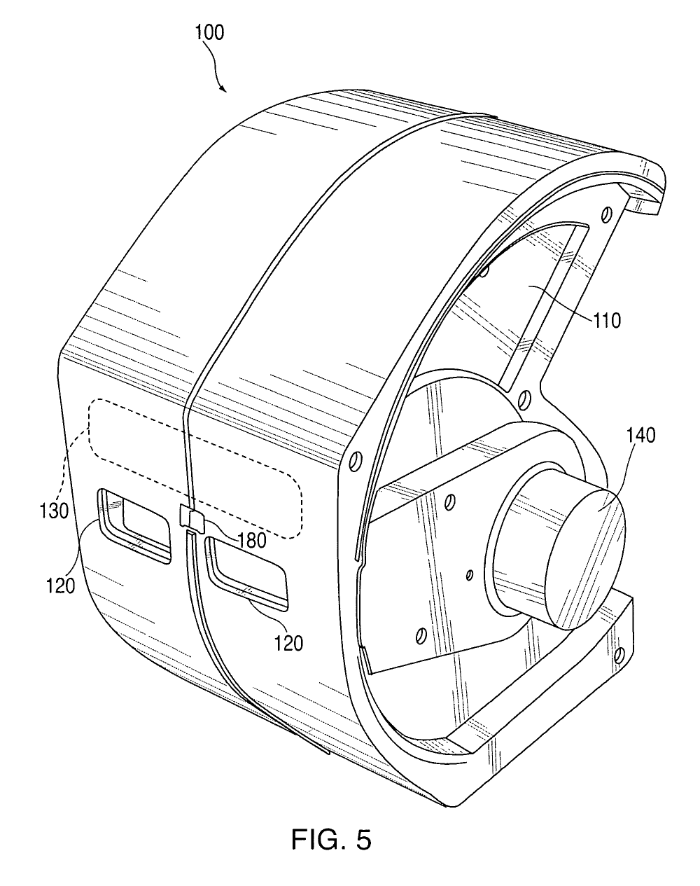

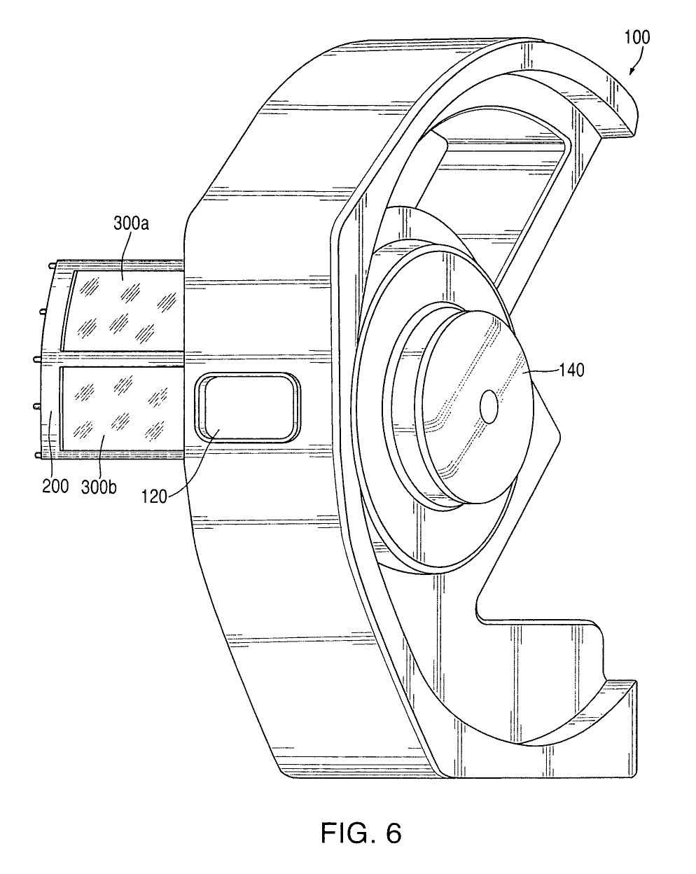

In an effort to help the consumer choose the magnification that best improves his farsightedness, a standard 14″ dimension is used as a guide for the distance between the eyes and the object being viewed. Turning now to FIGS. 1-12, the reading glass strength tester 100 of the present invention is contained within a standalone housing 110. The housing 110 preferably has a flat base so that it may rest on a table, counter or other flat surface. Alternatively, the reading glass strength tester 100 may also be incorporated into a point-of-sale display as with existing devices known to those skilled in the art. The housing and other parts of the tester are preferably constructed of sturdy plastic, although other suitable materials may be chosen.

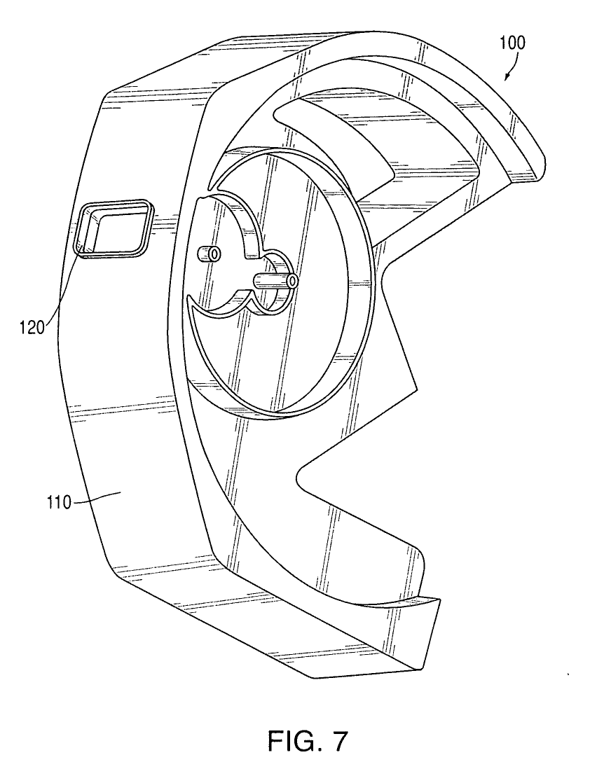

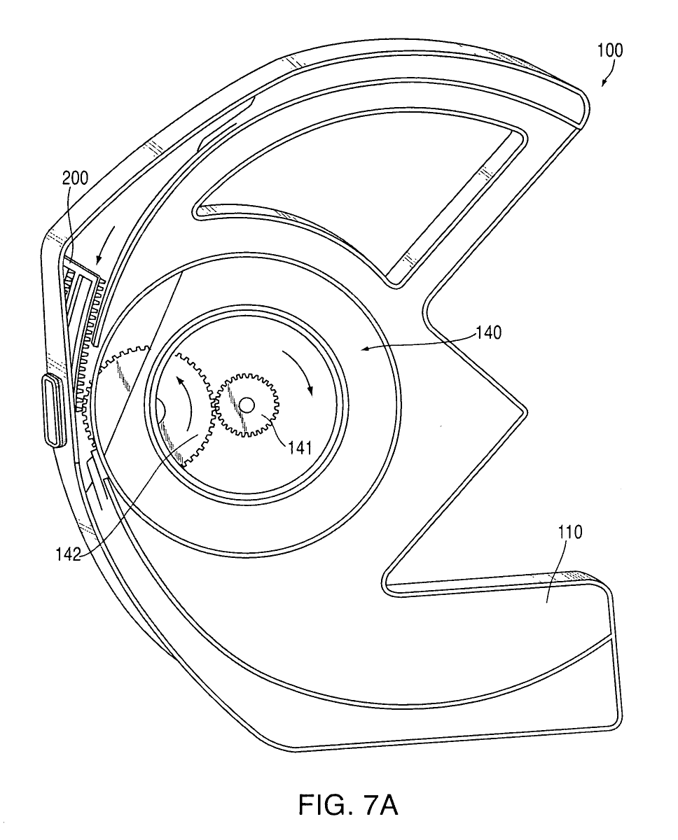

The front of the housing 110 has two viewports 120 located just below a forehead rest area 130 (indicated by dotted lines in FIGS. 1, 4 and 5). Behind the viewports 120 are a series of magnifying lenses 300 that can be moved in front of the ports 120 by turning knobs 140, located on each side of the housing 110. As shown in FIGS. 6, 8a, 9a and 10, the lenses 300 are secured in a lens carrier 200 that travels along a track 150 inside the housing 110. As shown in FIG. 7A, rotational motion of knobs 140 is transmitted via gears and pinions 141, 142 into curvilinear motion of lens carrier 200 along track 150 via rack 205. Turning the knobs in one direction causes the lens carrier to move in one direction; turning the knobs in the opposition direction causes the lens carrier to move in the other direction.

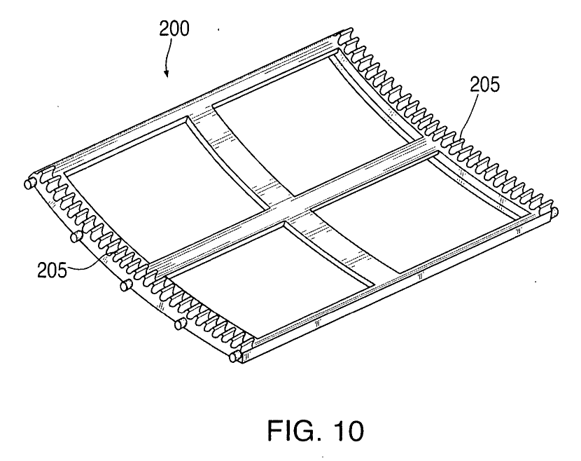

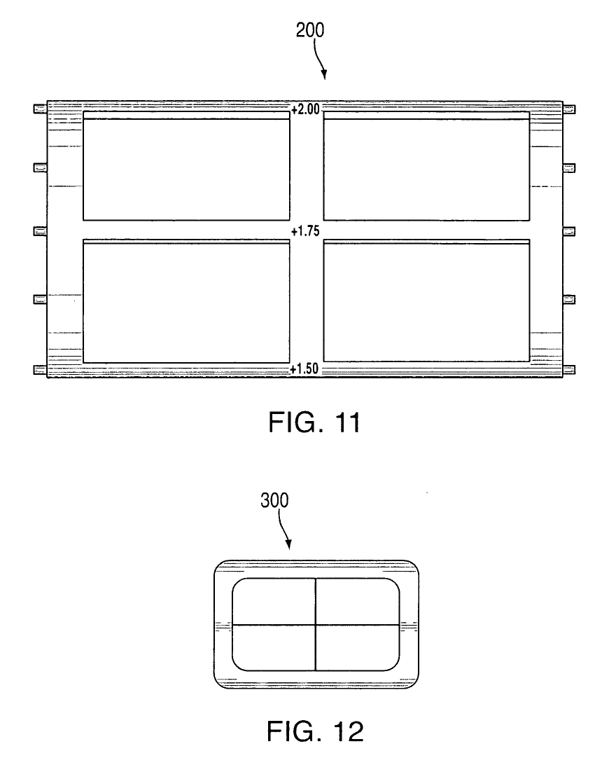

In one preferred embodiment, knobs 140 turn together so that lens carrier 200 advances both the left and right magnifying lenses 300 simultaneously. As shown in FIGS. 10 and 11, this may be accomplished by having a single lens carrier 200 that contains left and right lenses of equal magnification side by side to one another. The corresponding diopter is indicated visually on the center of the carrier 200. In an alternative embodiment, each knob 140 may be independently geared to a corresponding lens carrier 200. Rotation of the knob 140 in either direction causes the lens carrier 200 to move, but in this embodiment, the left knob causes the left lens carrier to move, while the right knob causes the right lens carrier to move. In this manner, a consumer can determine whether each eye requires a lens of the same or different strength.

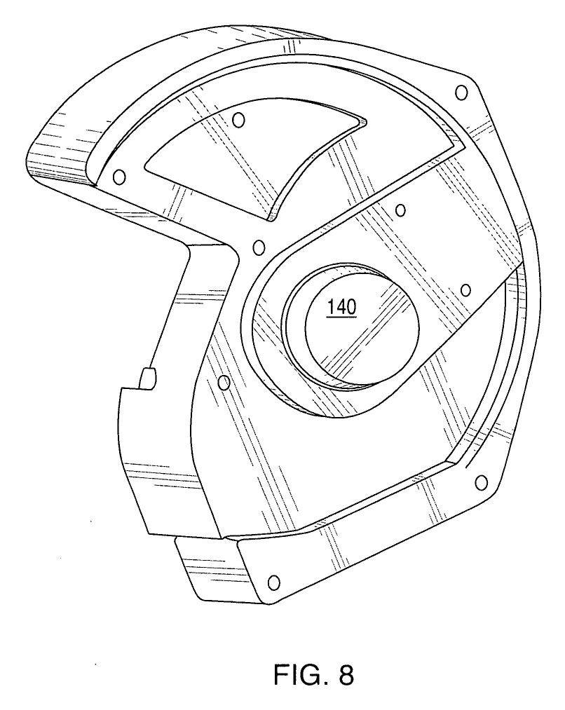

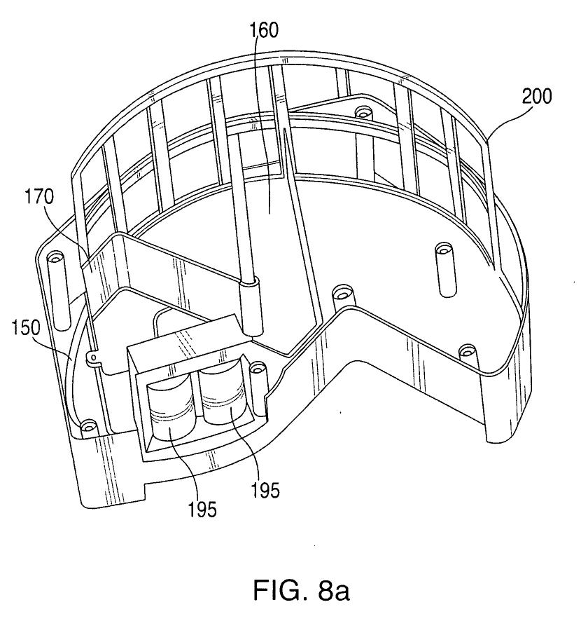

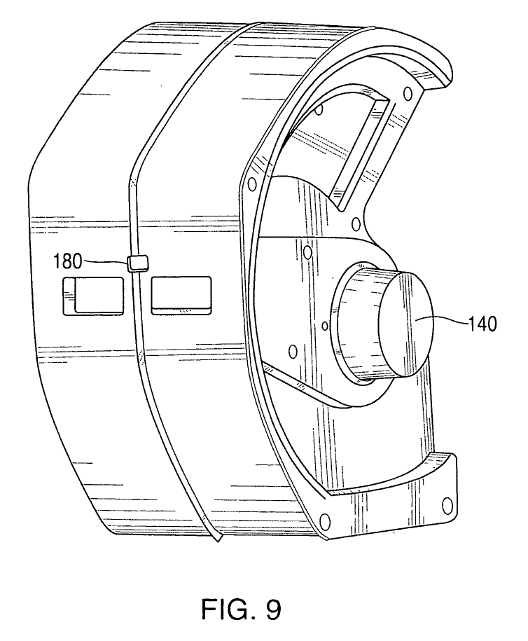

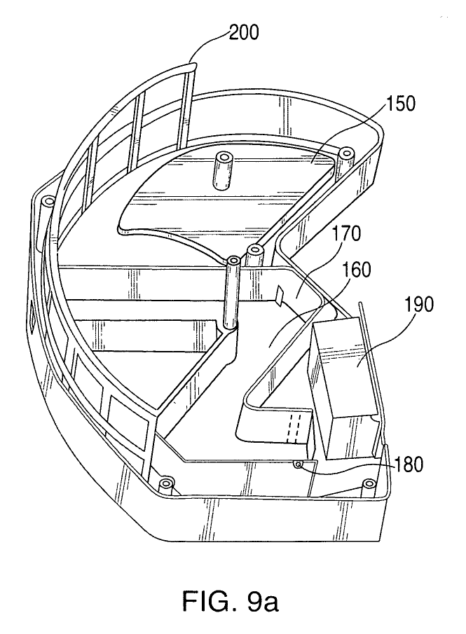

Turning to FIGS. 8a and 9a, visual indicia, comprising newsprint-style type on a reading surface (not shown) is located near the distal end of channel 180 and viewed at a distance of approximately 14″ from the viewports 120 by means of an indirect view path 160 using mirrors 170. Due to the non-linear view path 160, the reading glass strength tester of the present invention can be considerable smaller than existing devices. Although illustrated here with in a preferred two mirror embodiment, one skilled in the art will appreciate that any number of mirrors may be employed.

The reading surface with printed material is illuminated by means of battery operated light emitting diodes (LEDs) (not shown) or such other low power light sources known to those skilled in the art. The light source is preferably located behind the reading surface, although it may also illuminate the reading surface from in front, above, below or to either side, so long as it does not impair the image. When a button (not shown) is depressed, the light source is activated for a short period of time. The light source is preferably provided with a timer that turns off the light after a predetermined period of time. Alternatively, the light source can be set so that it turns on and off with each press of the button. In another embodiment, the light may be set to stay on only so long as the button is depressed. The light source may be powered by battery pack 190 containing one or more batteries 195, or by other power sources means well known to those skilled in the art (e.g. a solar cell, AC power, transformer, etc.). The light source is connected to the power source by electrical wiring, soldering or other such material known to those skilled in the art. In a preferred embodiment where a battery pack is employed as the power source, housing 110 is preferably provided with an access panel 195 to provide access to the power and light source (e.g. for changing the battery, LED(s), bulb(s), etc.). The power source may also be located remotely, that is, away from the unit in a fixture, cabinet, outlet, etc.

With the reading surface illuminated, the consumer can look through the view ports 120 and rotate the knobs 140 in one direction or the other to move the lens carrier 200 along tracks 150 to bring different power magnifying lenses 300 into the view path behind view ports 120. The consumer continues this process to determine which magnifying lenses are best for reading at this distance. After making this determination, the consumer can identify the diopter corresponding to the lens 300 by looking through diopter window 180 for a diopter (e.g. +1.00, +1.25, +1.50, +1.75, etc.) indicated on lens carrier 200 (see FIG. 11). The consumer is then free to chose a pair of reading glasses of the appropriate strength.

It will be apparent to those skilled in the art that various modifications and variations can be made in the present invention without departing from the spirit or scope of the invention. Thus, it is intended that the present invention covers the modifications and variations of this invention that come within the scope of any claims and their equivalents.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

1. A reading glass strength self-tester, said tester comprising:
a housing, wherein said housing incorporates a viewport in the front of said housing, a plurality of pairs of magnifying lens of different diopter ratings mounted behind said viewport, a means for advancing said lenses, at least one window incorporated into the front of said housing displaying said diopter ratings of said lenses, at least one mirror located behind said lenses mounted at an angle with respect to said viewport, a reading surface mounted to said housing located at an angle with respect to said at least one mirror, a non-ambient light source mounted inside said housing to illuminate said reading surface, a power source connected to said light source by electrical means and a means for turning on said power source and said light source.

2. The reading glass strength self-tester of claim 1, wherein said housing is portable.

3. The reading glass strength self-tester of claim 1, wherein said housing is made of plastic.

4. The reading glass strength self-tester of claim 1, wherein the light source is mounted behind said reading surface.

5. The reading glass strength self-tester of claim 1, wherein the power source is contained within said housing.

6. The reading glass strength self-tester of claim 5, wherein the power source and light source are accessible through an access panel.

7. The vision reading glass strength self-tester of claim 1, wherein the power source is solar power.

8. The reading glass strength self-tester of claim 1, wherein the power source is a battery.

9. The reading glass strength self-tester of claim 1, wherein the reading surface is about 14 inches along a non-linear view path.

10. The reading glass strength self-tester of claim 1, wherein the pairs of magnifying lenses move independently of each other with each lens having its own diopter rating associated with it.

11. A reading glass strength self-tester, said tester comprising:
a housing, wherein said housing incorporates a viewport in the front of said housing, a plurality of pairs of magnifying lens of different diopter ratings mounted behind said viewport, a means for advancing said lenses, at least one window incorporated into the front of said housing displaying said diopter ratings of said lenses, at least one mirror located behind said lenses mounted at an angle with respect to said viewport, a reading surface mounted to said housing located at an angle with respect to said at least one mirror, a non-ambient light source mounted inside said housing to illuminate said reading surface, a power source connected to said light source by electrical wire, a means for turning on said power source and said light source and a timer for turning off the light source after a predetermined time.

12. The reading glass strength self-tester of claim 11, wherein the timer is powered by the power source.

13. Reading glass strength self-tester of claim 11, wherein the timer is mechanically powered by the means for turning on said power source.

14. A reading glass strength self-tester, said tester comprising:
a lens carrier system mounted inside a housing comprising a plurality of pairs of magnifying lenses of different diopter ratings affixed to at least one flexible lens carrier, a track mounted inside said housing whereby the at least one carrier travels, and a means for advancing the at least one carrier along the track that is rotatably connected to said at lease one lens carrier.

15. The reading glass strength self-tester of claim 14, wherein said lens carrier system advances said lenses in alignment with a viewport incorporated into said housing.

16. The reading glass strength self-tester of claim 14, wherein said lens carrier moves along said at least one track via a rack.

17. The reading glass strength self-tester of claim 16, wherein said at least one track is rotatably connected to knobs protruding outwardly from said housing by at least one gear and pinion.

18. The reading glass strength self-tester of claim 14, wherein said knobs move independently of each other.

19. The reading glass strength self-tester of claim 14, wherein the diopter ratings are imprinted on said at least one lens carrier.
